(19) United States
(12) Patent Application Publication
(10) Pub. No.: US 2022/0047155 A1
(43) Pub. Date: Feb. 17, 2022

(54) Suppression Assessment Scoring and Mitigation Panel

(71) Applicant: University of Cincinnati, Cincinnati, OH (US)

(72) Inventor: Joseph F Clark, Cincinnati, OH (US)

(73) Assignee: University of Cincinnati, Cincinnati, OH (US)

(21) Appl. No.: 17/400,843

(22) Filed: Aug. 12, 2021

Related U.S. Application Data
(60) Provisional application No. 63/064,647, filed on Aug. 12, 2020.

(51) Int. Cl. A61B 3/024

(52) U.S. Cl. CPC A61B 3/024

(57) ABSTRACT

A method of diagnosing suppression in a subject is disclosed. The method involves prompting the subject to engage in three neurological assessments and recording the results from each test. The neurological assessments are a Brock String test, a Bates Field Splitters test, and a Thumb Thing test. The results of these tests are used to calculate a singular score that indicates the presence of suppression in the subject. In one embodiment, the method further includes using the results of the tests to diagnose the subject with near suppression, far suppression, or both.

### CROSS REFERENCE TO RELATED APPLICATIONS

The application claims priority to U.S. Provisional Application Ser. No. 63/064,647, filed Aug. 12, 2020, which application is hereby incorporated by reference in its entirety.

### TECHNICAL FIELD

The present invention relates to methods to diagnose suppression.

### BACKGROUND OF THE INVENTION

Fatigue induced suppression is common and often undiagnosed neurophysiologic condition that can cause tunnel vision. At the University of Cincinnati, 15% of athletes tested have fatigue induced suppression following practices and games. This type of tunnel vision is where the brain and eyes fatigue and turn off one eye. They eye is not blind per se, rather the brain is failing to take in that information thereby losing peripheral vision, losing visual information, and having tunnel vision. The person will not feel it occurring but will be prone to decreased performance and increased risks. A need still exists for improved methods to quickly and easily diagnose fatigue induced suppression.

### SUMMARY OF THE INVENTION

The present invention is a method of diagnosing suppression in a subject. The method involves prompting the subject to engage in three neurological assessments and recording the results from each test. The neurological assessments are a Brock String test, a Bates Field Splitters test, and a Thumb Thing test. The results of these tests are used to calculate a singular score that indicates the presence of suppression in the subject. In one embodiment, the method further includes using the results of the tests to diagnose the subject with near suppression, far suppression, or both.

In another embodiment, the method further includes the step of mitigating suppression if it is identified in the subject. The mitigation includes one or more of the following steps: a) notifying the subject that they have suppression, b) having the subject rest; and c) checking for two eye activity using a Thumb Thing two point fixation test. In one embodiment, the mitigation includes all three steps.

In one embodiment, the singular score is used to produce two possible results, either “no suppression” (a “green” score”) or “suppression indicated” (a “yellow,” “orange” or “red score”). In another embodiment, if suppression is indicated, the singular score is used to further identify the category of suppression from the following options: a) some concerns of suppression (a “yellow” score); b) concerns of suppression (an “orange” score); and c) substantial concerns of suppression (a “red” score).

In another embodiment of the present invention, a method of diagnosing suppression in a subject is disclosed. The method involves prompting the subject to engage in the following neurological assessments and recording the results from each test:

1) a Brock String test, where a score of 0 is assigned if the subject sees one bead and one string, a score of 1 is assigned if the subject sees one bead and two strings, and a score of 2 is assigned if the subject sees two strings converge or cross near the bead;
2) a Bates Field Splitters test, where a score of 0 is assigned if the subject sees one color, a score of 1 is assigned if the subject sees two colors and identifies them in the correct positions, and a score of 2 is assigned if the subject sees two colors and identifies them in the opposite positions;
3) a Thumb Thing test, where a score of 0 is assigned if the subject sees one thumb and one far object, a score of 1 is assigned if the subject sees two thumbs and the two thumbs are on either side of one far object, and a score of 2 is assigned if the subject sees two far objects on either side of one thumb; and adding the scores of these tests to produce a singular score that indicates the presence of suppression in the subject. In this embodiment, a singular score of 5-6 indicates no suppression, a singular score of 3-4 indicates some concerns of suppression, a singular score of 1-2 indicates concerns of suppression, and a singular score of 0 indicates substantial concerns of suppression

### DETAILED DESCRIPTION OF THE INVENTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

Certain terminology is used in the following description for convenience only and is not limiting. As used herein, “absence of suspicion of neurological injury” means that there is negligible clinical evidence that a diagnosis of injury can be made.

Similar reference numerals will be utilized throughout the application to describe similar or the same components of each of the preferred embodiments of the implant described herein and the descriptions will focus on the specific features of the individual embodiments that distinguish the particular embodiment from the others

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The present invention involves a novel method of diagnosing suppression. The invention uses a multiple test panel, which is important since suppression is not binary. There are multiple types and manifestations of suppression. Thus, one test is not likely to cover all the suppression types. Those skilled in the art would be able to list and describe other tests to assess various suppression types. The common types of suppression are: suppression, intermittent suppression, alternating suppression, near suppression, far suppression, field specific suppression, and combinations thereof. As a simple example of combined suppression, military service members were seen with induced alternating-intermittent-near-far suppression. This occurred because they used night vision goggles and set the goggles focal length to near for one eye and far for the other eye. When in the night vision goggles, they would suppress one or the other eye based on the need to see near or far. The goggles training induced the suppression. The suppression alternated based on the focus length needed at the time. The significance is that the conditions can dictate the type of suppression necessitating the use of multiple test panel as described in this invention.

The present invention uses a platform of validated methods to diagnose suppression that is simple and easy to teach as well as deploy to aid in preventing decrease in athlete performance or a fall in combat effectiveness due to this type of tunnel vision. In one embodiment, the present invention uses a quick and easy battery of tests to diagnose tunnel vision in athletes. This scoring method can also be implemented to maintain combat effectiveness. The present invention delivers methods to quantitatively diagnose fatigue induced tunnel vision and provides mitigation strategies. The overall goal is to be able to sustain performance under fatigue and stress conditions and to mitigate fatigue induced tunnel vision.

In one embodiment, the present invention involves a combination of three tests that produce a quantitative result that contains clinically relevant and actionable information. The present invention uses a simple 3-point panel to assess suppression from any cause. This 3-point panel can identify suppression. More specifically, it can identify the type and severity of the suppression. The three tests within this panel include (1) Brock string, (2) Bates Field Splitters, and (3) Thumb Thing. Individually, the three methods of the panel produce a limited and uncorroborated evaluation. However, the present invention concerns the surprising discovery that when used together, they form a unique methodology to cover suppression assessment.

### The Tests

The three tests used for the present invention tests are the Brock string, Bates Field Splitters, and Thumb Thing test. To pass the three tests both eyes must be working at both a near and far distance. The results are three parts. 1. Near suppression. 2. Far suppression. 3. Coordination of the two eyes to come in and out normally. The totality of the tests take less than two minutes to perform. Near suppression is when the eyes are dwelling on a near object and an eye suppresses. Near suppression can be assessed with thumb thing when looking at the thumb. Far suppression is when the eyes are dwelling on a far object and one of the eyes are suppressing. Far suppressing can be assessed with the Bates Field Splitters when looking at something far and the person only sees one color card.

Brock String. The brock string assesses the eye's ability to converge to a point and focus on that point. The result is two parts. 1. Two eyes being used—up close suppression assessment. 2. Two eyes coordinated together. In result 1, the two eyes are working or are “on” in that they are not suppressing. This is when there are two strings seen because each eye perceives the string from a slightly different orientation. Result 2, the eyes are coordinated. This is when the two strings converge onto one bead. When the vergence is before or after the bead that means the eyes are not coordinated onto the one bead. Thus over converging (strings cross before the bead) or under converging (strings cross after the bead) indicate the eyes are not coordinated. Converging to the bead (strings cross at the bead) indicates the eyes are well coordinated.

A Brock string comprises a line, such as a string, and one or more visual targets mounted on the string, such as beads. When a subject is looking at the bead on the string and the string is being held between the eyes coming straight out (ortho is the medical term), the person should see 1 bead in focus. If they see one bead and one string they would get a score of 0. If they see 1 bead and 2 strings, they are not likely to be suppressing up close and they would get 1 point. If they see the two strings converge or cross near the bead (substantially less than 3 inches) they get a score of 2 points. This test is assessing near suppression with a near target.

TABLE 1
Brock String Scores

| Result | Score |
|---|---|
| Subject sees one bead and one string | 0 |
| Subject sees one bead and two strings | 1 |
| Subject sees two strings converge or | 2 |
| cross near the bead (at less than 3 inches) | |

Bates Field Splitters. The Bates Field Splitters are two colored cards that provide two pieces of information. 1. Two eyes are being used—at a distance suppression. 2. The eyes process the different colors on the different sides of the body correctly. It is an assessment of a full field of vision.

When a subject is wearing or has the two color cards in front of their eyes and they are looking far away, typically more than 20 feet, they should be aware of the cards in front of their face. If they see one color they could be suppressing and would get a score of 0. If they claim they see two colors when looking far and they identify the correct colors they would get 1 point. If a person sees 2 colors and the colors do crossover, they would receive 2 points.

Note, the cross over of the colors reflects normal visual processing that ensures both eyes and bilateral brain regions are functioning. Lack of cross over suggests a possible deficiency in the processing in the brain concerning visual input. This test is assessing far suppression using a near target. It can also be used to assess near suppression by looking at a near target but is not scored in this paradigm.

TABLE 2
Bates Field Splitters Scores

| Result | Score |
|---|---|
| Subject sees one color | 0 |
| Subject sees two colors and identifies them | 1 |
| in the correct positions | |
| Subject sees two colors and identifies them | 2 |
| in the opposite positions (“cross over”) | |

Thumb Thing. The thumb thing is not a device. It is a method to assess near and far suppression and ocular motor coordination. Thumb thing is a method that can be done by a trainer, service member, medic or other personnel quickly. It is hard to game the adduction abduction assessment. The first part of the thumb thing can be done in groups. For example, a quick check of a squad before action. In this situation, a medic tells a group to all look at an object more than 20 feet away—e.g., a flag. Hold up 1 thumb at the level of the flag. Keep both eyes open, cover the flag with your thumb. Do you still see the flag, do you see two thumbs? Anyone who says NO to either question, gets further evaluations.

When a person looks at an object first for a few moments and brings one thumb up to the level of the object he/she should see two thumbs. Note, the thumb should be arm's length away, also the far object should be more than 20 feet away and no bigger than a basketball. If they see 1 thumb and one far object they would get a score of 0. If they see two thumbs and they put the two thumbs on either side of the far object, confirm the two thumbs are on either side of the far object, they get 1 point. Next, have them look at the thumb. If they see two of the far objects on either side of the one thumb they get a score of 2 points. This test is assessing near and far suppression. Near suppression with a far target object and far suppression with a near target object.

TABLE 3
Thumb Thing Scores

| Result | Score |
|---|---|
| Subject sees one thumb and one far object | 0 |
| Subject sees two thumbs and the two thumbs | 1 |
| are on either side of one far object | |
| Subject sees two far objects on either side of | 2 |
| one thumb | |

Note: Near/Far, and oculomotor can also be added assessments for additional information.

### Grading for the Three Tests:

Scores from the three tests are totaled. The total scores are assigned a color as follows:

0=Red

1 to 2=Orange

3 to 4=Yellow

5 to 6=Green

Additional Info: There are additional points of information that can be added when assessing suppression with the panel of the present invention. These notes should be taken down and can be added to the assessment paradigm and/or used for designing mitigation strategies. Some observations to be noted are (1) Symmetric eye movement with using Thumb Thing, (2) Good convergence with using Brock String, and (3) Near and far suppression absent when using Thumb Thing.

In one embodiment, the result of this panel of tests is a binary result of:

1) No—not suppressing. No concern regarding performance decrement (Green=good), or
2) Yes—suppressing. There is concern for tunnel vision and that tunnel vision is graded to a severity scale of (yellow, orange or red). In some embodiments, the color scheme has corresponding recommendations for mitigating performance decrement and performance sustainment.

In another embodiment, the result of this panel of tests is one of four options:

1) Green—no concerns. No changes in performance or performance expectations.
2) Yellow—Some concerns. Repeat tests in a prescribed time frame to assess tunnel vision progression.
3) Orange—Concerns. Use prescribed mitigation methods (we have several including a two minute mitigation method and similar treatments). Re-evaluate as needed.
4) Red—Substantial concerns. Rest and prescribed treatments. Re-evaluate as needed.

### Actionable Information

The method of the present invention can be used in a variety of situations. If suppression is indicated during a practice, the individual can engage in a suppression mitigation protocol. If suppression is indicated during a game, combat, competition, etc., the results of the present method can be used to make safety-oriented decisions on next steps. In an acute situation where suppression is noted during activity, such as play or combat, the main mitigation strategy is for the person to be aware that it is happening. Another mitigation tool is to check for two eye activity using a Thumb Thing two point fixation test, where the two point fixation is looking at the far object (point 1) and the near object such as the thumb (point 2). If the player is able to rest that could be considered as well.

If suppression is noted during training activities, a more thorough examination to assess why the suppression is occurring and mitigation strategies to prevent suppression can be engaged. If the cause of the suppression is ocular motor fatigue, then ocular motor conditioning could be engaged. This information depends upon conditions.

Information caveat. Suppression is one form of tunnel vision. It is when the brain turns off one eye and relies on the sensory information from the other eye. This cuts the visual information to the brain. The brain is in effect resting by having less visual information coming in. However, that “rest” leads to tunnel vision and a concomitant increase in risks on the battlefield. There are other kinds of tunnel vision (cortical tunnel vision, visual neglect etc). The present invention is mainly addressing suppression as the cause of tunnel vision and the concomitant treatments therein.

### Materials and Training

The “equipment” needed to do the tests are: two color cards (playing cards or 3 by 5 cards), a string with one or two beads on it. The beads can be the size of a water-bottle lid. The beads could be a colored binder clip or a button. Thus it is a very small weight and volume that could fit in an envelope or pocket. It could also be rigged from existing equipment.

Train the Trainer. It takes about 10 minutes to demonstrate the tests and demonstrate normal responses. It takes approximately 20 minutes to explain the scoring to a reasonably trained clinical practitioner (athletic trainer, experienced combat medic, doctor etc). To train the instructors on how to train the field personnel to do the diagnostic assessment may take about 2 hours. It can be done in a classroom with groups of people and variations of the equipment (formal equipment as well as makeshift equipment) to cover a range of situations. This training will also provide demonstrations of normal, abnormals, as well as spotting the abnormals.

### Mitigation

Once a quantitative assessment has been made using the suppression panel of the present invention, that information can be used to formulate a treatment plan. The suppression assessment panel provides actionable information. There could be an acute treatment plan or an extended mitigation plan. Again, those skilled in the art will make decisions concerning treatment plan types, modalities and timing.

Some considerations one might contemplate concerning treatment plans are the modalities. The Brock string could be used to treat near or far suppression. This could be done with single or multi bead strings, with thick and thin strings of varying lengths. The distance of the bead(s) can be changed to address the type of distance of the suppression.

In one embodiment, the Bates field splitters are used as a passive treatment for suppression. A subject wears the glasses and regularly ensures correct card and color visualization during various (preferably safe) activities. Another passive treatment that is useful for the present invention is stroboscopic glasses or strobe lights. Those skilled in the art will have methods and protocols for treating suppression with stroboscopic effects.

Treating suppression with two color filters and matched color sheets or computer screens is another useful treatment for suppression mitigation. Suppression treatment with the Bates field splitters can be accomplished by having a person wear the glasses and do tasks at a distance where they regularly verify they are seeing two colors. Computer based suppression treatment can be accomplished by using a reading task where the letters and numbers are different colors and the person is wearing two color glasses. The two color glasses require the two eyes to work such that letters and words can be read. This method can be applied to treating suppression assessed by our suppression panel. A putative goal here is that one eye can only see one set of alpha numerics based on color of the filter and the other eye can see the other alpha numerics. Thus, a task such as saccades would require the use of both eyes thereby treating suppression.

In general, optometrists and neuro trained specialists (experienced and trained in these arts) will be experienced and familiar with suppression treatment options. The list above is not meant to be exhaustive or restrictive. It is somewhat a descriptive list of what and how many might consider treating suppression once found with our suppression pane. Binocular vision training and treatment is often used for suppression treatment and is well known to clinical practitioners such as optometrists, ophthalmologists and neurologic specialists.

### Military Relevance

Tunnel vision is a common risk in combat, tactical, athletic, stress and fatigue situations. For neuro trained specialists tunnel vision is a catch phrase for several neurologic situations. The current proposal is designed to diagnose and mitigate one type of tunnel vision referred to as suppression. Suppression is the “turning off” of one eye and it can occur for numerous reasons. Regardless of the reason for the suppression it is analogous to temporary blindness in one eye and can put military service members at risk for losing situational awareness, field of view, peripheral vision and degraded performance. The present invention provides a quick and low tech method that can be used by deployed medics to diagnose and treat this type of tunnel vision. It will improve combat effectiveness and decrease risks on the battlefield.

### EXAMPLES

Example 1. Six subjects were assessed for suppression using all three of the tests of the present invention. The results were totaled and scored (see Table 4).

TABLE 4

| | Bates Field | Brock | Thumb | | |
|---|---|---|---|---|---|
| Subject | Splitters | String | Thing | Total | Color |
| 1 | 2 | 2 | 1 | 5 | Green |
| 2 | 2 | 2 | 2 | 6 | Green |
| 3 | 2 | 2 | 2 | 6 | Green |
| 4 | 2 | 2 | 2 | 6 | Green |
| 5 | 1 | 2 | 0 | 3 | Yellow |
| 6 | 1 | 0 | 2 | 3 | Yellow |

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A method of diagnosing suppression in a subject comprising prompting the subject to engage in the following neurological assessments and recording the results from each test:
a. a Brock String test,
b. a Bates Field Splitters test;
c. a Thumb Thing test; and
using the results of these tests to calculate a singular score that indicates the presence of suppression in the subject.

2. The method of claim 1 wherein the singular score is used to produce two possible results:
a. no suppression; or
b. suppression indicated.

3. The method of claim 2 wherein if suppression is indicated, the singular score is used to further identify the category of suppression from the following options:
a. some concerns of suppression;
b. concerns of suppression;
c. substantial concerns of suppression.

4. The method of claim 1 further comprising using the results of the tests to diagnose the subject with near suppression, far suppression, or both.

5. The method of claim 1 further comprising the step of mitigating suppression if it is identified in the subject, wherein the mitigation comprises one or more of the following steps:
a. notifying the subject that they have suppression,
b. having the subject rest; and
c. checking for two eye activity using a Thumb Thing two point fixation test.

6. The method of claim 5 wherein the mitigation comprises all of the following steps:
a. notifying the subject that they have suppression,
b. having the subject rest; and
c. checking for two eye activity using a Thumb Thing two point fixation test.

7. The method of claim 1 further comprising the step of mitigating suppression if it is identified in the subject, wherein the mitigation comprises having the subject wear Bates Field Splitters glasses and perform tasks at a distance where they regularly verify that they are seeing two colors.

8. The method of claim 1 further comprising the step of mitigating suppression if it is identified in the subject, wherein the mitigation comprises having the subject wear Bates Field Splitters glasses and performing a reading task where the letters and numbers are different colors.

9. A method of diagnosing suppression in a subject consisting of prompting the subject to engage in the following neurological assessments and recording the results from each test:
a. a Brock String test,
b. a Bates Field Splitters test;
c. a Thumb Thing test; and
using the results of these tests to calculate a singular score that indicates the presence of suppression in the subject.

10. A method of diagnosing suppression in a subject comprising prompting the subject to engage in the following neurological assessments and recording the results from each test:
a. a Brock String test, where a score of 0 is assigned if the subject sees one bead and one string, a score of 1 is assigned if the subject sees one bead and two strings, and a score of 2 is assigned if the subject sees two strings converge or cross near the bead;
b. a Bates Field Splitters test, where a score of 0 is assigned if the subject sees one color, a score of 1 is assigned if the subject sees two colors and identifies them in the correct positions, and a score of 2 is assigned if the subject sees two colors and identifies them in the opposite positions;
c. a Thumb Thing test, where a score of 0 is assigned if the subject sees one thumb and one far object, a score of 1 is assigned if the subject sees two thumbs and the two thumbs are on either side of one far object, and a score of 2 is assigned if the subject sees two far objects on either side of one thumb; and
adding the scores of these tests to produce a singular score that indicates the presence of suppression in the subject; wherein a singular score of 5-6 indicates no suppression, a singular score of 3-4 indicates some concerns of suppression, a singular score of 1-2 indicates concerns of suppression, and a singular score of 0 indicates substantial concerns of suppression.

11. The method of claim 10 further comprising using the results of the tests to diagnose the subject with near suppression, far suppression, or both.

12. The method of claim 10 further comprising the step of mitigating suppression if it is identified in the subject, wherein the mitigation comprises one or more of the following steps:
a. notifying the subject that they have suppression,
b. having the subject rest; and
c. checking for two eye activity using a Thumb Thing two point fixation test.

13. The method of claim 12 wherein the mitigation comprises all of the following steps:
a. notifying the subject that they have suppression,
b. having the subject rest; and
c. checking for two eye activity using a Thumb Thing two point fixation test.

14. The method of claim 10 further comprising the step of mitigating suppression if it is identified in the subject, wherein the mitigation comprises having the subject wear Bates Field Splitters glasses and perform tasks at a distance where they regularly verify that they are seeing two colors.

15. The method of claim 10 further comprising the step of mitigating suppression if it is identified in the subject, wherein the mitigation comprises having the subject wear Bates Field Splitters glasses and performing a reading task where the letters and numbers are different colors.
